(19) United States
(12) Patent Application Publication
(10) Pub. No.: US 2009/0061005 A1
(43) Pub. Date: Mar. 5, 2009

(54) Paliperidone Polymorphs

(75) Inventors: Uday Rajaram Bapat, Kandivali (IN); Jayamani Munusamy, Asthinapuram (IN); Sivaji Ravisaravanan, K. Pudur (IN); Vigneshwara Ravisankar, Gandhi Nagar (IN); Jon Valgeirsson, Hafnarfjorour (IS)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/194,878

(22) Filed: Aug. 20, 2008

Related U.S. Application Data
(60) Provisional application No. 60/981,136, filed on Oct. 19, 2007.

(30) Foreign Application Priority Data
Aug. 21, 2007 (IN) 1867/CHE/2007

(51) Int. Cl. A61K 9/14; C07D 471/04; A61K 31/519

(52) U.S. Cl. 424/489; 544/282; 514/259.41

(57) ABSTRACT

Described herein are novel polymorphic forms of paliperidone, processes for preparing the novel forms and use thereof. Also provided are processes for the preparation of novel polymorphic forms of paliperidone and the use thereof in the preparation of pharmaceutical compositions.

### CROSS REFERENCE TO RELATED APPLICATION

This application claims the benefit of priority to Indian provisional application No. 1867/CHE/2007, filed on Aug. 21, 2007, and U.S. Provisional Application No. 60/981,136, filed on Oct. 19, 2007, which are incorporated herein by reference.

### FIELD OF THE DISCLOSURE

Described herein are novel polymorphic forms of Paliperidone, processes for preparing and use thereof. Also provided are processes for the preparation of novel polymorphic forms of Paliperidone and the use thereof in the preparation of pharmaceutical compositions.

### BACKGROUND

U.S. Pat. Nos. 4,804,663 and 5,158,952 disclose a variety of 3-piperidinyl-1,2-benzisoxazole derivatives, processes for their preparation, pharmaceutical compositions comprising the derivatives, and methods of use thereof. These compounds have long-acting antipsychotic properties and are useful in the treatment of warm-blooded animals suffering from psychotic diseases. Among them, paliperidone, (±)-3-[2-[4-(6-fluoro-1,2-benzisoxazol-3-yl)-1-piperidinyl]ethyl]-6,7,8,9-tetrahydro-9-hydroxy-2-methyl-4H-pyrido[1,2-a]pyrimidin-4-one, is an antipsychotic agent and indicated for the both acute (short-term) and maintenance (long-term) treatment of schizophrenia. Paliperidone has the following structural formula:

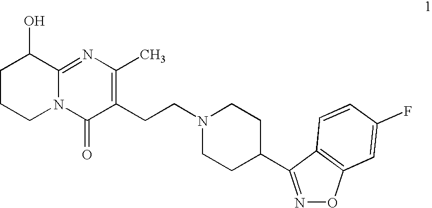

Processes for the preparation of paliperidone and related compounds are disclosed in U.S. Pat. Nos. 5,158,952; 5,254,556; 5,688,799; and 6,320,048.

According to U.S. Pat. No. 5,158,952 (hereinafter referred to as the '952 patent), paliperidone can be prepared by the reaction of 3-(2-chloroethyl)-6,7,8,9-tetrahydro-9-hydroxy-2-methyl-4H-pyrido[1,2-a]-pyrimidin-4-one with 6-fluoro-3-(4-piperidinyl)-1,2-benzisoxazole in the presence of a base, in a reaction inert solvent and optionally in the presence of a phase transfer catalyst. The inert solvents include water; an aromatic solvent, e.g., benzene, methylbenzene, dimethylbenzene, chlorobenzene, methoxybenzene, and the like; a C₁₋₆ alkanol, e.g., methanol, ethanol, 1-butanol and the like; a ketone, e.g., 2-propanone, 4-methyl-2-pentanone, and the like; an ester, e.g., ethyl acetate, gamma.-butyrolactone, and the like; an ether, e.g., 1,1′-oxybisethane, tetrahydrofuran, 1,4-dioxane, and the like; a dipolar aprotic solvent, e.g., N,N-dimethylformamide, N,N-dimethylacetamide, dimethylsulfoxide, pyridine, 1,3-dimethyl-3,4,5,6-tetrahydro-2(1H)-pyrimidinone, 1,3-dimethyl-2-imidazolidinone, 1,1,3,3-tetramethylurea, 1-methyl-2-pyrrolidinone, nitrobenzene, acetonitrile, and the like; or a mixture thereof. The bases include inorganic bases such as, for example, an alkali metal or an earth alkaline metal carbonate, hydrogen carbonate, hydroxide, oxide, carboxylate, alkoxide, hydride or amide, e.g., sodium carbonate, sodium hydrogen carbonate, potassium carbonate, sodium hydroxide, calcium oxide, sodium acetate, sodium methoxide, sodium hydride, sodium amide, and the like, or an organic base such as, for example, a tertiary amine, e.g., N,N-diethylethanamine, N-(1-methylethyl)-2-propanamine, 4-ethylmorpholine, 1,4-diazabicyclo[2.2.2]octane, pyridine and the like. The phase transfer catalysts include trialkylphenylmethylammonium, tetraalkylammonium, tetraalkylphosphonium, tetraarylphosphonium halide, hydroxide, hydrogen sulfate, and the like. The reaction mixture containing paliperidone obtained is then subjected to evaporation, and the oily residue is extracted with trichloromethane followed by water washings. The organic layer is dried, filtered and evaporated followed by column chromatographic purifications over silica gel using a mixture of trichloromethane and methanol. The pure fractions are collected and the eluent is evaporated. The resulting residue was crystallized from 2-propanone. After cooling, the precipitated product was filtered off, washed with a mixture of 2-propanol and 2,2′-oxybispropane and recrystallized from 2-propanol to produce paliperidone.

PCT Publication No. WO 08/021,342 A2 (hereinafter referred to as the '342 application) discloses six crystalline forms of paliperidone (Forms I-VI), and characterizes them by powder X-ray diffraction (P-XRD) and solid state ¹³C NMR. According the '342 application, crystalline Form I is characterized by an X-ray powder diffraction pattern having peaks expressed as 2-theta at about 10.1, 12.4, 14.3, 17.0, 17.2, 18.9, 21.9, 24.8 and 26.2±0.2 degrees; crystalline Form II is characterized by an X-ray powder diffraction pattern having peaks expressed as 2-theta at about 8.1, 10.3, 13.1, 13.7, 14.6, 14.9, 16.2, 18.6, 19.2, 20.0, 20.6, 22.0, 24.6, 25.0, 27.9 and 31.2±0.2 degrees; crystalline Form III is characterized by an X-ray powder diffraction pattern having peaks expressed as 2-theta at about 10.8, 14.1, 15.9, 16.8, 17.2, 19.1, 21.1, 21.7 and 25.8±0.2 degrees; crystalline Form IV is characterized by an X-ray powder diffraction pattern having peaks expressed as 2-theta at about 10.3, 12.1, 15.4, 19.8, 20.7, 23.9 and 33.1±0.2 degrees; crystalline Form V is characterized by an X-ray powder diffraction pattern having peaks expressed as 2-theta at about 9.7, 10.9, 14.0, 15.8, 16.3, 21.1, 21.5 and 26.0±0.2 degrees; and crystalline Form VI is characterized by an X-ray powder diffraction pattern having peaks expressed as 2-theta at about 5.8, 8.4, 9.5, 11.6, 19.1, 20.3, 23.7, 24.7 and 31.7±0.2 degrees.

Polymorphism is defined as “the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and infrared spectrometry (IR).

Solvent medium and mode of isolation play very important role in obtaining a polymorphic form over the other.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Hence, there remains a need in the art for novel and stable polymorphic forms of paliperidone.

### SUMMARY

We have now surprisingly and unexpectedly discovered three novel polymorphic forms of paliperidone with adequate stability and good dissolution properties.

In one aspect, provided herein is a novel and stable hydrate form of paliperidone having water content of about 2.5-4% by weight, designated as paliperidone polymorphic Form A2, characterized by at least one, and specifically all, of the following properties:

i) an IR spectrum substantially in accordance with FIG. 1;

ii) an IR spectrum having absorption bands at about 3527, 3388, 3153, 2955, 2707, 2653, 1641, 1614, 1536 and 1131±2 cm⁻¹, substantially as depicted in FIG. 1;

iii) a DSC thermogram substantially in accordance with FIG. 2; and

iv) a TGA thermogram substantially in accordance with FIG. 3.

In another aspect, further included herein is a process for preparing the highly pure and stable polymorphic Form A2 of paliperidone having water content of about 2.5-4% by weight.

In another aspect, included herein is a novel and stable polymorphic form of paliperidone, designated as paliperidone polymorphic Form A3, characterized by an X-ray powder diffraction pattern having peaks expressed as 2-theta angle positions at about 5.04, 7.76, 9.62, 10.20, 10.75, 12.02, 13.06, 13.47, 13.89, 14.39, 15.36, 15.66, 16.18, 17.83, 19.07, 19.67, 21.06, 21.41, 22.31, 23.75, 25.00, 25.82, 26.89, 27.2, 30.83, 32.86 and 37.37±0.2 degrees 2-theta.

In another aspect, included herein is a process for preparing the highly pure and stable polymorphic Form A3 of paliperidone.

In yet another aspect, provided herein is a novel and stable polymorphic form of paliperidone, designated as paliperidone polymorphic Form A4, characterized by an X-ray powder diffraction pattern having peaks expressed as 2-theta angle positions at about 7.40, 8.15, 9.65, 10.25, 10.78, 12.39, 13.07, 13.80, 14.53, 14.91, 15.71, 16.20, 18.64, 19.14, 19.97, 20.44, 20.66, 21.07, 21.48, 21.99, 24.62, 25.00, 25.83, 27.95, 28.53, 30.72 and 31.15±0.2 degrees 2-theta.

In another aspect, included herein is a process for preparing the highly pure and stable polymorphic Form A4 of paliperidone.

In another aspect, provided herein are pharmaceutical compositions comprising a therapeutically effective amount of any one of the paliperidone polymorphic forms or mixtures thereof as disclosed herein, and one or more pharmaceutically acceptable excipients.

In another aspect, included herein are pharmaceutical compositions comprising the polymorphic forms of paliperidone prepared according to the processes disclosed herein in any of their embodiments, and one or more pharmaceutically acceptable excipients.

In yet another aspect, included herein is a process for preparing a pharmaceutical formulation comprising combining any one of the polymorphic forms of paliperidone prepared according to processes disclosed herein in any of their embodiments, with one or more pharmaceutically acceptable excipients.

In another aspect, the substantially pure polymorphic forms of paliperidone disclosed herein are employed in pharmaceutical compositions, wherein 90 volume-percent of the particles (D₉₀) have a size of less than or equal to about 400 microns, specifically less than or equal to about 300 microns, more specifically less than or equal to about 200 microns, still more specifically less than or equal to about 100 microns, and most specifically less than or equal to about 15 microns.

### BRIEF DESCRIPTION OF THE DRAWINGS

FIG. 1 is a characteristic infra red (IR) spectrum of paliperidone polymorphic Form A2.

FIG. 2 is a characteristic Differential Scanning Calorimetric (DSC) thermogram of paliperidone polymorphic Form A2.

FIG. 3 is characteristic Thermogravimetric Analysis (TGA) thermogram of paliperidone polymorphic Form A2.

FIG. 4 is a characteristic powder X-ray diffraction (XRD) pattern of paliperidone polymorphic Form A3.

FIG. 5 is a characteristic Infra red (IR) spectrum of paliperidone polymorphic Form A3.

FIG. 6 is a characteristic Differential Scanning Calorimetric (DSC) thermogram of paliperidone polymorphic Form A3.

FIG. 7 is a characteristic powder X-ray diffraction (XRD) pattern of paliperidone polymorphic Form A4.

FIG. 8 is a characteristic Infra red (IR) spectrum of paliperidone polymorphic Form A4.

FIG. 9 is a characteristic Differential Scanning Calorimetric (DSC) thermogram of paliperidone polymorphic Form A4.

### DETAILED DESCRIPTION OF THE INVENTION

According to one aspect, provided herein is a novel and stable hydrate form of paliperidone having water content of about 2.5-4% by weight, designated as paliperidone polymorphic Form A2, characterized by at least one, and specifically all, of the following properties:

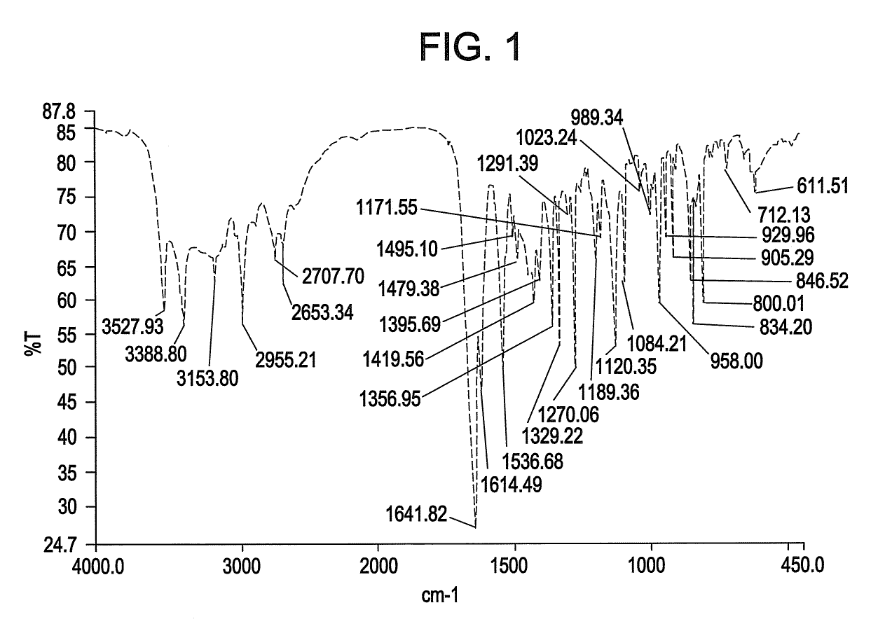

i) an IR spectrum substantially in accordance with FIG. 1;

ii) an IR spectrum having absorption bands at about 3527, 3388, 3153, 2955, 2707, 2653, 1641, 1614, 1536 and 1131±2 cm⁻¹ substantially as depicted in FIG. 1;

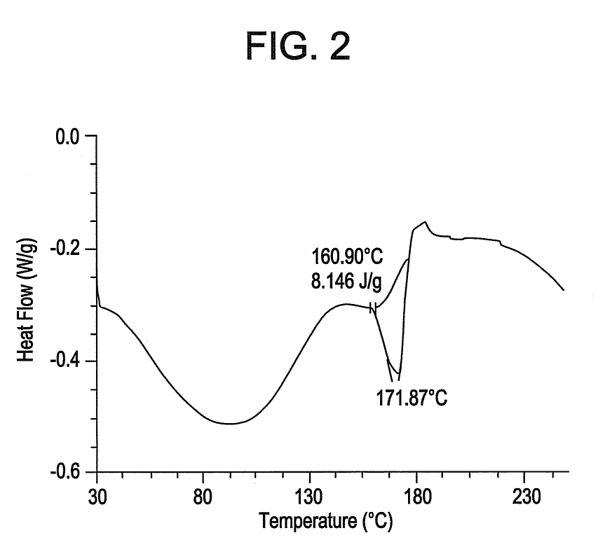

iii) a DSC thermogram substantially in accordance with FIG. 2; and

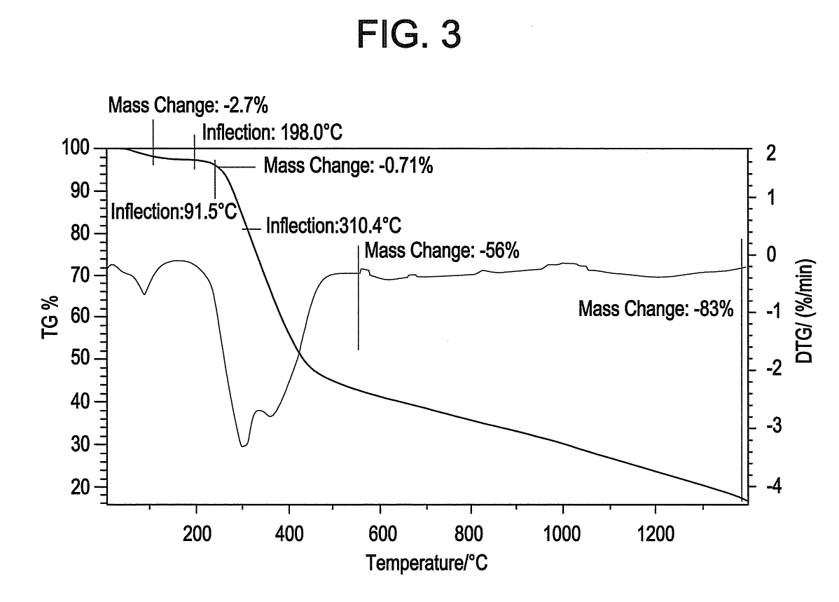

iv) a TGA thermogram substantially in accordance with FIG. 3.

According to another aspect, a process is provided for preparation of a stable and substantially pure paliperidone polymorphic Form A2 having water content of about 2.5-4% by weight, which comprises:

a) providing a suspension of paliperidone in an alcoholic solvent;

b) combining the suspension with hydrochloric acid to form a first solution;

c) substantially removing the solvent from the first solution to provide a solid product;

d) dissolving the solid product in water to form a second solution;

e) neutralizing the second solution obtained with base to provide a reaction mass containing paliperidone; and

f) recovering the polymorphic Form A2 of paliperidone having water content of about 2.5-4% by weight from the reaction mass.

The process can produce polymorphic Form A2 of paliperidone in substantially pure form.

The term “substantially pure paliperidone polymorphic Form A2” refers to the paliperidone polymorphic Form A2 having purity greater than about 99%, specifically greater than about 99.5%, and more specifically greater than about 99.9% (measured by HPLC).

The paliperidone polymorphic Form A2 (having water content of about 2.5-4% by weight) is stable, consistently reproducible and has good flow properties, and which is particularly suitable for bulk preparation and handling, and so, the novel paliperidone polymorphic Form A2 is suitable for formulating paliperidone. Moreover, the polymorphic Form A2 of paliperidone is useful intermediate in the preparation of paliperidone polymorphic Forms A3 and A4 in high purity.

Exemplary alcoholic solvents used in step-(a) include, but are not limited to, C₁₋₈ straight or branched chain alcohol solvents, such as, for example, methanol, ethanol, n-propanol, isopropanol, n-butanol, isobutanol, tert-butanol and the like, and mixtures thereof. Specific alcoholic solvents are methanol, ethanol and isopropanol, and most specifically methanol.

In one embodiment, step-(a) of providing a suspension of paliperidone includes suspending paliperidone in the alcoholic solvent while stirring at a temperature below boiling temperature of the solvent used. In one embodiment, the suspension is stirred at a temperature of about 15° C. to about 80° C. for at least 15 minutes, and more specifically at about 20° C. to about 50° C. from about 30 minutes to about 5 hours.

Paliperidone used as starting material in step-(a) can be prepared by the processes described in the U.S. Pat. No. 5,158,952.

The step-(a) of providing a suspension of paliperidone may also include suspending a form of paliperidone in the alcoholic solvent or obtaining an existing suspension from a previous processing step.

The hydrochloric acid used in step-(b) may be in the form of aqueous hydrochloric acid, in the form of hydrogen chloride gas, or hydrogen chloride dissolved in an alcoholic solvent. The alcoholic solvents used for dissolving hydrogen chloride gas or hydrogen chloride include, but are not limited to, C₁₋₈ alcohol solvents such as methanol, ethanol, isopropanol, and the like, and mixtures thereof. A specific alcohol solvent is methanol.

Combining of the suspension with hydrochloric acid in step-(b) is done in a suitable order, for example, the suspension is added to the hydrochloric acid, or alternatively, the hydrochloric acid is added to the suspension. The addition is, for example, carried out drop wise or in one portion or in more than one portion. The addition is specifically carried out at a temperature of below about 50° C. for at least 15 minutes, and more specifically at a temperature of about 15° C. to about 35° C. from about 20 minutes to about 2 hours. After completion of addition process, the resulting mass is stirred for at least 20 minutes, more specifically for about 30 minutes to about 4 hours, at a temperature of about 20° C. to about 35° C.

The solution obtained in step-(b) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing the product by removing charcoal. In one embodiment, finely powdered carbon is an active carbon.

Removal of solvent in step-(c) is accomplished by, for example, substantially complete evaporation of the solvent, concentrating the solution and filtering the solid under inert atmosphere. Alternatively, the solvent is removed by evaporation. Evaporation is achieved, for example, at sub-zero temperatures by the lyophilization or a freeze-drying technique. The solution may be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a solid.

The distillation process is, for example, performed at atmospheric pressure or reduced pressure. Specifically, the solvent is removed at a pressure of about 760 mm Hg or less, more specifically at about 400 mm Hg or less, still more specifically at about 80 mm Hg or less, and most specifically at about 30 to about 80 mm Hg.

In one embodiment, the dissolution in step-(d) is carried out at a temperature of below about 100° C., more specifically at about 25° C. to about 80° C., and still more specifically at about 25° C. to about 60° C. After completion of the dissolution, the resulting solution is optionally cooled to about 10° C. to about 20° C.

The base used for neutralization in step-(e) is an inorganic base. In one embodiment, an aqueous solution of inorganic base is used. Exemplary inorganic bases include, but are not limited to, ammonia; hydroxides, carbonates and bicarbonates of alkali or alkaline earth metals. Specific alkali metals are lithium, sodium and potassium, and more specifically sodium and potassium. Specific alkaline earth metals are calcium and magnesium, and more specifically magnesium. Specific inorganic bases are ammonia, sodium hydroxide, calcium hydroxide, magnesium hydroxide, potassium hydroxide, lithium hydroxide, sodium carbonate, potassium carbonate, lithium carbonate, and more specifically ammonia, sodium hydroxide and potassium hydroxide.

In one embodiment, the neutralization reaction in step-(e) is carried out at a temperature of below about 50° C. for at least 15 minutes, and more specifically at a temperature of about 10° C. to about 35° C. from about 30 minutes to about 4 hours. Specifically, the pH of the solution in step-(e) is adjusted at about 6.5-9, and more specifically at about 7.5-8.5 with a suitable base.

The recovering in step-(f) is carried out by conventional techniques known in the art such as filtration, filtration under vacuum, decantation, and centrifugation and dried to obtain substantially pure paliperidone polymorphic Form A2. In one embodiment, paliperidone polymorphic Form A2 is isolated by filtration employing a filtration media of, for example, a silica gel or celite.

The pure paliperidone polymorphic Form A2 obtained by above process is optionally further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

The pure paliperidone polymorphic Form A2 obtained by above process has water content of about 2.5-4% w/w, which is stable and consistently reproducible, and the moisture could not be removed even after extended drying for 12 hours at about 80° C. under vacuum.

The total purity of the paliperidone polymorphic Form A2 obtained by the process disclosed herein is of greater than about 99%, specifically greater than about 99.5%, and more specifically greater than about 99.9% as measured by HPLC.

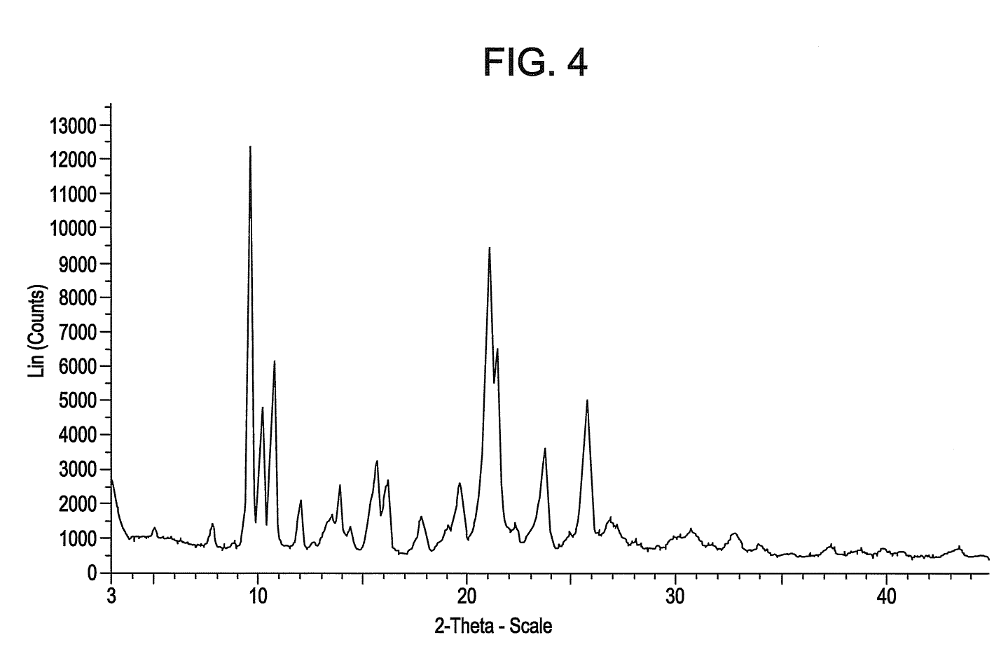

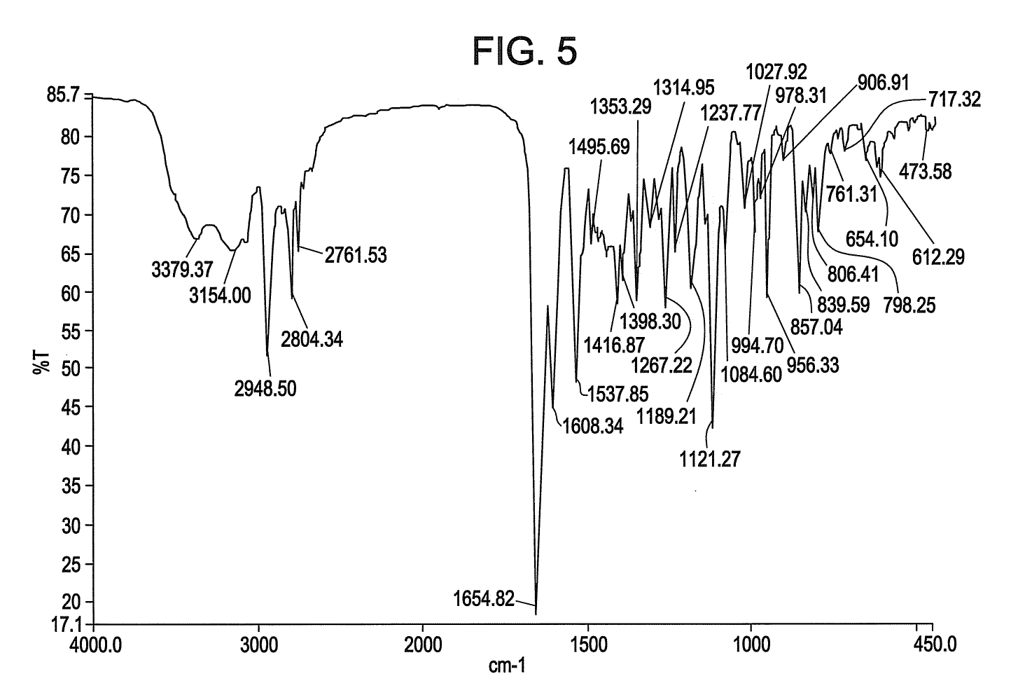

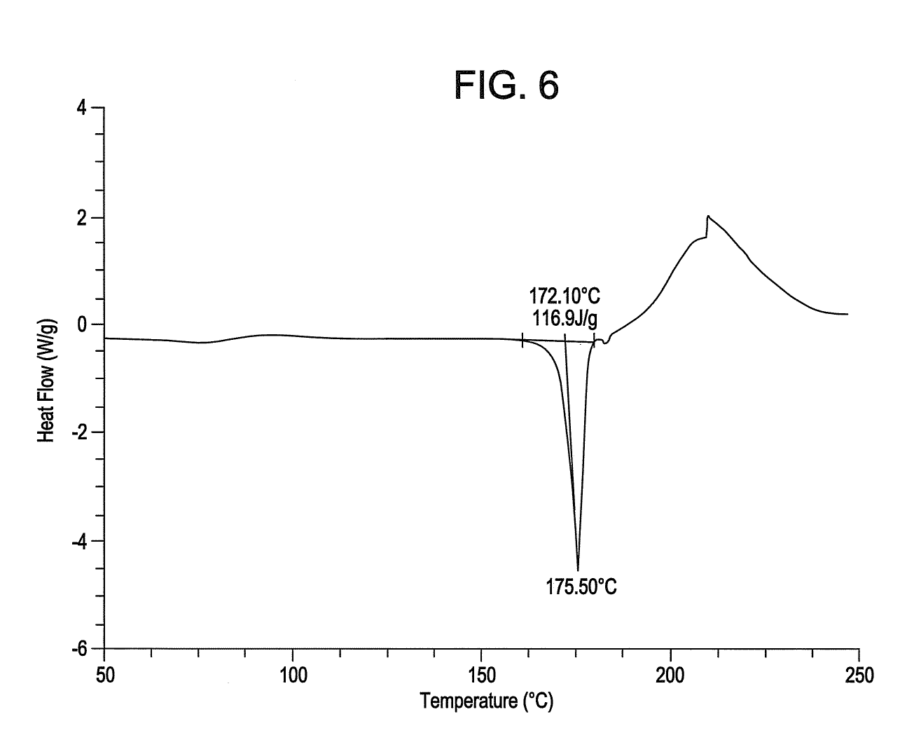

In another aspect, provided is a novel polymorphic form of paliperidone, designated as polymorphic Form A3, characterized by at least one, and specifically all, of the following properties:
i) a powder X-ray diffraction pattern substantially in accordance with FIG. 4;
ii) a powder X-ray diffraction pattern having peaks at about 5.04, 7.76, 9.62, 10.20, 10.75, 12.02, 13.06, 13.47, 13.89, 14.39, 15.36, 15.66, 16.18, 17.83, 19.07, 19.67, 21.06, 21.41, 22.31, 23.75, 25.00, 25.82, 26.89, 27.2, 30.83, 32.86 and 37.37±0.2 degrees 2-theta substantially as depicted in FIG. 4;
iii) a powder X-ray diffraction pattern having additional peaks at about 12.64, 27.58, 28.12, 30.01, 31.24, 31.75, 32.43, 34.02, 38.81, 39.86, 40.76 and 43.46±0.2 degrees 2-theta substantially as depicted in FIG. 4;
iv) an IR spectrum substantially in accordance with FIG. 5;
v) an IR spectrum having absorption bands at about 2948, 2804, 2761, 1654, 1608, 1537, 1267 and 1121±2 cm⁻¹; and/or
vi) a DSC thermogram substantially in accordance with FIG. 6.

In one embodiment, a process is provided for preparation of a stable and substantially pure paliperidone polymorphic Form A3, which comprises:

a) providing a suspension of paliperidone in water;

b) combining the suspension with an acid to form a clear solution;

c) neutralizing the clear solution obtained in step-(b) with a base to provide a reaction mass containing paliperidone; and

d) recovering polymorphic Form A3 of paliperidone from the reaction mass.

The process can produce polymorphic Form A3 of paliperidone in substantially pure form.

The term “substantially pure paliperidone polymorphic Form A3” refers to the paliperidone polymorphic Form A3 having purity greater than about 99%, specifically greater than about 99.5%, and more specifically greater than about 99.9% (measured by HPLC).

The paliperidone polymorphic Form A3 is stable, consistently reproducible and has good flow properties, and which is particularly suitable for bulk preparation and handling, and so, the novel paliperidone polymorphic Form A3 is suitable for formulating paliperidone. Moreover, the polymorphic Form A3 of paliperidone is useful intermediate in the preparation of paliperidone polymorphic Form A4 in high purity.

Step-(a) of providing a suspension of paliperidone includes suspending paliperidone in water under stirring at a temperature of below about 100° C. Specifically, the suspension is stirred at a temperature of about 15° C. to about 80° C. for at least 15 minutes, and more specifically at about 20° C. to about 50° C. from about 30 minutes to about 5 hours.

Paliperidone used as starting material in step-(a) can be prepared by the processes described in the U.S. Pat. No. 5,158,952.

The step-(a) of providing a suspension of paliperidone may also include suspending any form of paliperidone in water or obtaining an existing suspension from a previous processing step.

The acid used in step-(b) can be an organic or inorganic acid. In one embodiment, an aqueous solution of acid is used. Exemplary inorganic acids include, but are not limited to, hydrochloric acid, hydrobromic acid, sulfuric acid, phosphoric acid, nitric acid and the like. Exemplary organic acids include, but are not limited to, p-toluenesulfonic acid, methanesulfonic acid, benzenesulfonic acid, oxalic acid, acetic acid, propionic acid, trifluoroacetic acid, carbonic acid, succinic acid, citric acid, tartaric acid, benzoic acid, maleic acid, fumaric acid and the like. In one embodiment, the acid used in step-(b) is hydrochloric acid.

The combining of the suspension with acid in step-(b) is done in a suitable order, for example, the suspension is added to the acid, or alternatively, the acid is added to the suspension. The addition is carried out drop wise, in one portion, or in more than one portion. In one embodiment, addition is carried out at a temperature of below about 50° C. for at least 15 minutes, and more specifically at a temperature of about 15° C. to about 35° C. from about 20 minutes to about 2 hours. After completion of addition process, the resulting mass is stirred for at least 20 minutes, more specifically about 30 minutes to about 4 hours, at a temperature of about 20° C. to about 35° C.

The solution obtained in step-(b) is optionally subjected to carbon treatment. The carbon treatment is carried out by methods known in the art, for example by stirring the solution with finely powdered carbon at a temperature of below about 70° C. for at least 15 minutes, specifically at a temperature of about 40° C. to about 70° C. for at least 30 minutes; and filtering the resulting mixture through hyflo to obtain a filtrate containing the product by removing charcoal. In one embodiment, finely powdered carbon is an active carbon.

The base used for neutralization in step-(c) is an organic or inorganic base. In one embodiment, an aqueous solution of base is used. Exemplary organic bases include, but are not limited to, triethyl amine, dimethyl amine, tert-butyl amine, diisopropyl amine, dimethyl amine, monomethyl amine and diisopropyl ethyl amine. In one embodiment the base is an inorganic base. Exemplary inorganic bases include, but are not limited to, ammonia; hydroxides, carbonates and bicarbonates of alkali or alkaline earth metals. Specific alkali metals are lithium, sodium and potassium, and more specifically sodium and potassium. Specific alkaline earth metals are calcium and magnesium, and more specifically magnesium.

Specific inorganic bases are ammonia, sodium hydroxide, calcium hydroxide, magnesium hydroxide, potassium hydroxide, lithium hydroxide, sodium carbonate, potassium carbonate, lithium carbonate, and more specifically ammonia, sodium hydroxide and potassium hydroxide.

In one embodiment, the neutralization reaction in step-(c) is carried out at a temperature of below about 50° C. for at least 15 minutes, and more specifically at a temperature of about 10° C. to about 35° C. from about 30 minutes to about 4 hours. In one embodiment, the pH of the solution in step-(c) is adjusted at about 6.5-9, and more specifically at about 7.5-8.5 with a suitable base.

The recovering in step-(d) is carried out by conventional techniques known in the art such as filtration, filtration under vacuum, decantation, and centrifugation and dried to obtain substantially pure paliperidone polymorphic Form A3. In one embodiment, paliperidone polymorphic Form A3 can be isolated by filtration employing a filtration media of, for example, a silica gel or celite.

The pure paliperidone polymorphic Form A3 obtained by above process is optionally further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

The total purity of the paliperidone polymorphic Form A3 obtained by the process disclosed herein is of greater than about 99%, specifically greater than about 99.5%, and more specifically greater than about 99.9% as measured by HPLC.

In another embodiment, provided herein is a novel polymorphic form of paliperidone, designated as polymorphic Form A4, characterized by at least one, and specifically all, of the following properties:

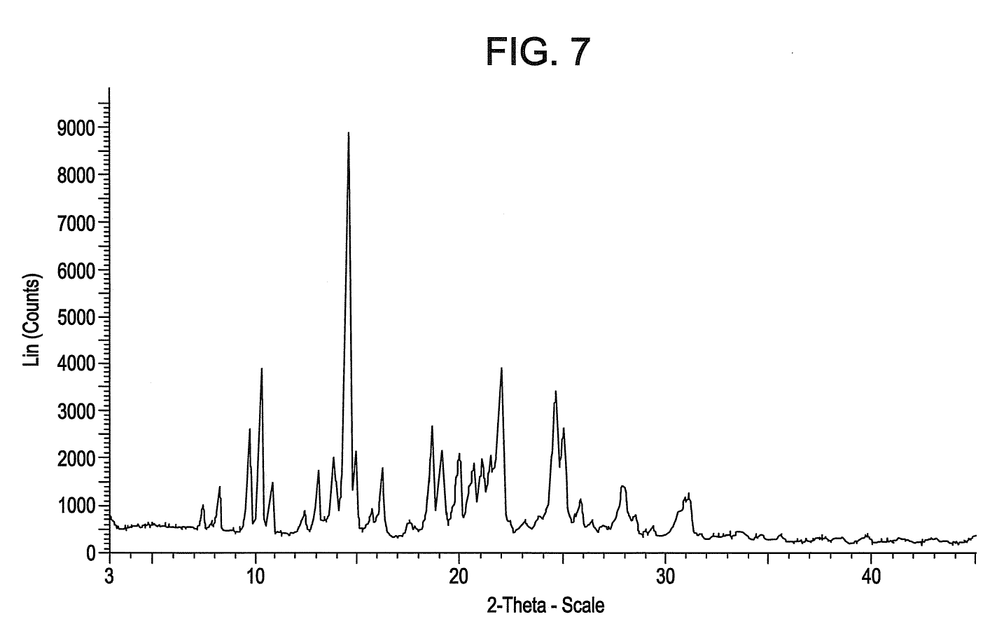

i) a powder X-ray diffraction pattern substantially in accordance with FIG. 7;

ii) a powder X-ray diffraction pattern having peaks at about 7.40, 8.15, 9.65, 10.25, 10.78, 12.39, 13.07, 13.80, 14.53, 14.91, 15.71, 16.20, 18.64, 19.14, 19.97, 20.44, 20.66, 21.07, 21.48, 21.99, 24.62, 25.00, 25.83, 27.95, 28.53, 30.72 and 31.15±0.2 degrees 2-theta substantially as depicted in FIG. 7;

iii) a powder X-ray diffraction pattern having additional peaks at about 7.82, 17.58, 17.85, 22.41, 23.15, 23.84, 26.36, 26.95, 29.07, 29.38, 33.60, 35.62, 38.58, 39.50, 39.80 and 42.84±0.2 degrees 2-theta substantially as depicted in FIG. 7;

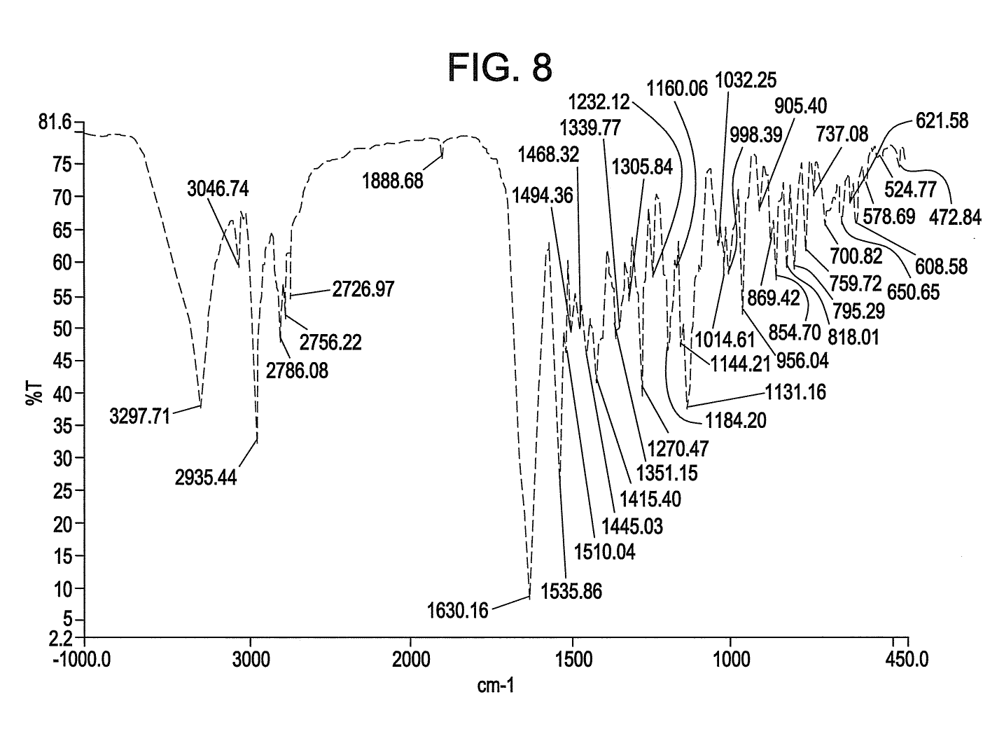

iv) an IR spectrum substantially in accordance with FIG. 8;

v) an IR spectrum having absorption bands at about 2935, 2786, 2756, 2726, 1630, 1535, 1270 and 1131±2 cm⁻¹; and/or

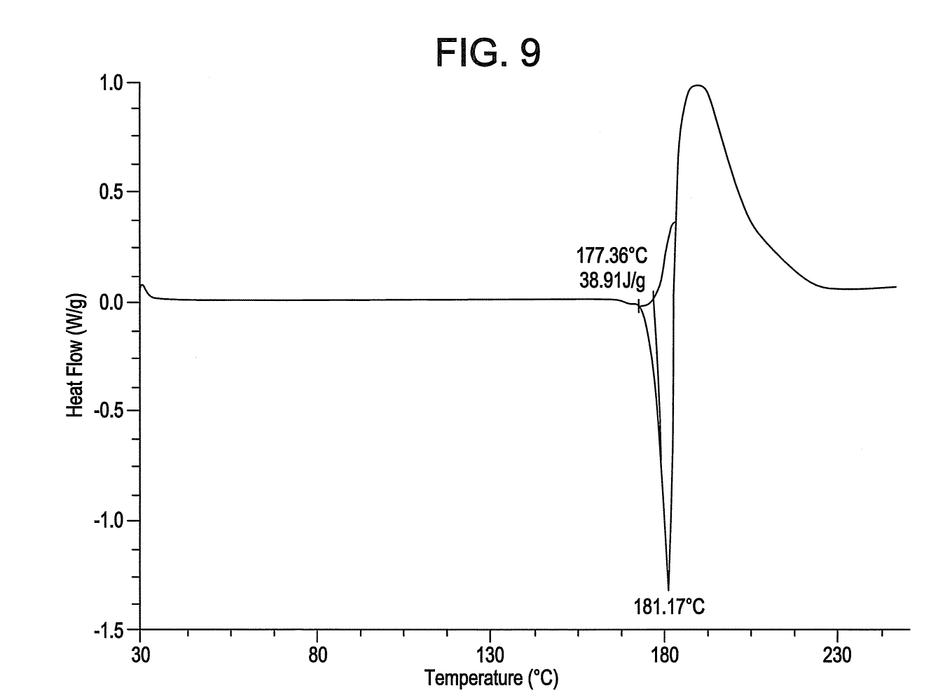

vi) a DSC thermogram substantially in accordance with FIG. 9.

In another aspect, a process is provided for preparation of a stable and substantially pure paliperidone polymorphic Form A4, which comprises:

a) suspending paliperidone polymorphic Form A3 in water to form a suspension;

b) heating the suspension obtained at a temperature above about 40° C. for a time period sufficient to convert the Form A3 into Form A4; and

c) recovering polymorphic Form A4.

The process can produce polymorphic Form A4 of paliperidone in substantially pure form.

The term “substantially pure paliperidone polymorphic Form A4” refers to the paliperidone polymorphic Form A4 having purity greater than about 99%, specifically greater than about 99.5%, and more specifically greater than about 99.9% (measured by HPLC).

The paliperidone polymorphic Form A4 is stable, consistently reproducible and has good flow properties, and which is particularly suitable for bulk preparation and handling, and so, the novel paliperidone polymorphic Form A4 is suitable for formulating paliperidone.

The paliperidone polymorphic Form A3 used as starting material in step-(a) may be obtained by the process disclosed herein above.

The suspension in step-(b) is specifically heated at a temperature of about 45° C. to about 80° C. for at least 30 minutes, and more specifically at a temperature of about 50° C. to about 70° C. from about 1 hour to about 8 hours.

The recovering in step-(c) is carried out by conventional techniques known in the art such as filtration, filtration under vacuum, decantation, and centrifugation and dried to obtain substantially pure paliperidone polymorphic Form A4. In one embodiment, paliperidone polymorphic Form A4 is isolated by filtration employing a filtration media of, for example, a silica gel or celite.

The pure paliperidone polymorphic Form A4 obtained by above process is optionally further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

The total purity of the paliperidone polymorphic Form A4 obtained by the process disclosed herein is of greater than about 99%, specifically greater than about 99.5%, and more specifically greater than about 99.9% as measured by HPLC.

In one embodiment, any one or a mixture of the substantially pure polymorphic forms of paliperidone (Form A2, Form A3 and Form A4) disclosed herein is used in pharmaceutical compositions, wherein 90 volume-percent of the particles (D₉₀) have a size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure polymorphic forms of paliperidone is achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect, there is provided pharmaceutical compositions comprising a therapeutically effective amount of paliperidone polymorphic forms disclosed herein and one or more pharmaceutically acceptable excipients.

In another embodiment, provided herein is a pharmaceutical composition comprising a therapeutically effective amount of any one or a mixture of the polymorphic forms of paliperidone disclosed herein, and one or more pharmaceutically acceptable excipients.

According to another aspect, there are provided pharmaceutical compositions comprising the polymorphic forms of paliperidone prepared according to processes disclosed herein and one or more pharmaceutically acceptable excipients.

According to another aspect, there is provided a process for preparing a pharmaceutical formulation comprising combining any one or a mixture of the polymorphic forms of paliperidone prepared according to processes disclosed herein, with one or more pharmaceutically acceptable excipients.

Yet another embodiment is directed to pharmaceutical compositions comprising at least a therapeutically effective amount of any one of the substantially pure polymorphic forms of paliperidone disclosed herein. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The polymorphic forms of paliperidone may also be administered as suppositories, ophthalmic ointments and suspensions, and parenteral suspensions, which are administered by other routes. The dosage forms may contain any one of the polymorphic forms of paliperidone as is or, alternatively, may contain any one of the polymorphic forms of paliperidone of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In another embodiment, there is provided pharmaceutical compositions comprising paliperidone polymorphic Form A2 and one or more pharmaceutically acceptable excipients.

In another embodiment, there is provided pharmaceutical compositions comprising paliperidone polymorphic Form A3 and one or more pharmaceutically acceptable excipients.

In another embodiment of the present invention, there is provided pharmaceutical compositions comprising paliperidone polymorphic Form A4 and one or more pharmaceutically acceptable excipients.

Capsule dosages, for example, contain the polymorphic forms of paliperidone within a capsule which may be coated with gelatin. Tablets and powders are optionally coated with an enteric coating. The enteric-coated powder form have coatings containing, for example, phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions may contain diluents such as cellulose-derived materials such as powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### EXPERIMENTAL

The HPLC purity was measured by high performance liquid chromatography by using a Waters, alliance 2695 HPLC system having dual wavelength UV detector under the following conditions:

| | |
|---|---|
| Column: | X-tera C18 (15 cm × 4.6 mm × 5μ) |
| Colunm oven temperature: | 30° C. |
| Detection: | 237 nm |
| Flow rate: | 1 ml/min |
| Injection volume: | 20 μl |
| Run time: | 35 min |
| Diluent: | MeOH:ACN:water 1:1:1 |

The following examples are provided to enable one skilled in the art to practice the invention and are merely illustrate the process of this invention. However, it is not intended in any way to limit the scope of the present invention.

### Examples

### Methods

The X-Ray powder diffraction was measured by an X-ray powder diffractometer equipped with a Cu-anode (λ=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2θ; step width=0.01579; and measuring time per step=0.11 second.

The FT-IR spectrum was measured by FT-IR SPECTRUM ONE—PERKINELMER equipped with a mid infrared detector—DTGS (deuterated triglycine sulphate) and an optical system collection over a range of 7800 to 370 cm⁻¹ with a best resolution of 0.5 cm⁻¹.

The DSC thermogram was measured by DSC Q20-TA equipped with Refrigerated cooling system (RCS40). The sample was analyzed using following instrument parameter: measuring range 30° C. to 300° C.; step with 10° C.

Thermogravimetric analysis (TGA) was performed with a NETZSCH STA 409C/CD instrument.

### Example 1

### Preparation of Polymorphic Form A2 of Paliperidone

Paliperidone (10 g) was suspended in methanol (100 ml) at 30-35° C. The suspension was followed by the addition of methanolic HCl (15 ml) to get a clear solution. The reaction mixture was stirred at 30-35° C. for 30-60 minutes and filtered. Methanol was removed from the filtrate under vacuum to get a residue. The residue was dissolved in water (100 ml). The resulting solution was cooled to 10-15° C. The pH of the solution was adjusted at about 7.5-8.0 at 10-15° C. by using aqueous ammonia to precipitate paliperidone. The resulting mass was stirred at 10-15° C. for about 2 hours and the solid was filtered. The solid obtained was washed with water (50 ml) and dried under high vacuum at 60-70° C. for about 12 hours to yield Form A2 of Paliperidone (Yield: 75%; HPLC Purity: 99.5%; Moisture content: 4% w/w).

### Example 2

### Preparation of Polymorphic Form A2 of Paliperidone

Paliperidone (2 g) was suspended in methanol (20 ml) at 30-35° C. Methanolic HCl (10 ml, 8.5% w/w) was added to the suspension at 30-35° C. followed by stirring to get a clear solution. Methanol was removed completely from the solution under vacuum to get a residue. The resulting residue was added to water (15 ml) followed by stirring to form a clear solution. The pH of the solution was adjusted at about 6.5-8.0 by using aqueous ammonia at 30-35° C. to precipitate paliperidone. The resulting solid was filtered, washed with water (10 ml) and dried under high vacuum at 60-70° C. for about 12 hours to yield Form A2 of Paliperidone (Yield: 1.5 g; HPLC Purity: 99.4%; Moisture content: 2.7% w/w).

### Example 3

### Preparation of polymorphic Form A3 of Paliperidone

Paliperidone (10 g) was suspended in water (100 ml) at 20-30° C. Suspension was followed by the addition of concentrated HCl (3.4 ml) to get a clear solution. The mixture was stirred at 20-30° C. for 30-60 minutes and filtered the solution. The pH of the filtrate was adjusted at about 7.5-8.0 by adding aqueous ammonia solution to precipitate paliperidone. The resulting mass was further stirred at 20-30° C. for 2 hours. The resulting solid was filtered, washed with water and then dried under high vacuum at 60-70° C. for 12 hours to yield Form A3 of Paliperidone (Yield: 67%, HPLC Purity: 99.43%).

### Example 4

### Preparation of Polymorphic Form A4 of Paliperidone

Paliperidone (10 g) was suspended in water (100 ml) at 20-30° C. suspension was followed by the addition of concentrated HCl (3.4 ml) to obtain clear solution. The mixture was stirred at 20-30° C. for 30-60 minutes and filtered. The pH of the filtered solution was adjusted at about 7.5-8.0 at 20-30° C. by adding aqueous ammonia over 30 minutes to precipitate paliperidone. The resulting mass was further stirred at 20-30° C. for 1 to 2 hours and the solid was filtered. The resulting solid was filtered, washed with water and then dried under high vacuum at 60-70° C. for 12 hours. The solid obtained was suspended in water (200 ml) to get slurry and then heated at 50-60° C. for 4 hours. The resulting solid was filtered and then dried under high vacuum at 60-70° C. for 12 hours to yield Form A4 of Paliperidone (Yield: 70%, HPLC Purity: 99.6%).

Unless otherwise indicated, the following definitions are set forth to illustrate and define the meaning and scope of the various terms used to describe the invention herein.

The term “polymorphic form” means a crystal modification that can be characterized by analytical methods such as X-ray powder diffraction, IR-spectroscopy, differential scanning calorimetry (DSC) or by its melting point.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” means a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The expression “pharmaceutically acceptable salt” means those salts which are, within the scope of sound medical judgment, suitable for use in contact with the tissues of humans and lower animals without undue toxicity, irritation, allergic response and the like, commensurate with a reasonable benefit/risk ratio, and effective for their intended use.

The term “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” means a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” means a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” means substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” means chemically inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” means agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” means substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” means a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g., Avicel™, carsium (e.g., Amberlite™, alginates, sodium starch glycolate, gums such as agar, guar, locust bean, karaya, pectin, tragacanth, combinations thereof and other such materials known to those of ordinary skill in the art.

The term “wetting agent” means a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

As used herein, D_{X} means that X percent of the particles have a diameter less than a specified diameter D. Thus, a D₉₀ of less than 300 microns means that 90 volume-percent of the micronized particles in a composition have a diameter less than 300 microns.

The term “micronization” means a process or method by which the size of a population of particles is reduced.

As used herein, the term “micron” or “elm” both mean “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

The term “water content” refers to the content of water based upon the Loss on Drying method as described in Pharmacopeial Forum, Vol. 24, No. 1, page 5438 (January-February 1998). The calculation of water content is based upon the percent of weight that is lost by drying. For Form A2 the term “water content” refers to the content of water based upon a TGA measurement.

By “substantially pure” is meant having purity greater than about 99%, specifically greater than about 99.5%, and more specifically greater than about 99.9% measured by HPLC.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A hydrated polymorphic Form A2 of paliperidone having water content of about 2.5 to about 4% by weight.

2. The compound of claim 1, characterized by at least one of the following properties:
i) an IR spectrum substantially in accordance with FIG. 1;
ii) an IR spectrum having absorption bands at about 3527, 3388, 3153, 2955, 2707, 2653, 1641, 1614, 1536 and 1171±2 cm⁻¹ substantially as depicted in FIG. 1;
iii) a DSC thermogram substantially in accordance with FIG. 2; and
iv) a TGA thermogram substantially in accordance with FIG. 3.

3. A process for the preparation of paliperidone polymorphic Form A2 of claim 1, comprising:
a) providing a suspension of paliperidone in an alcoholic solvent;
b) combining the suspension with hydrochloric acid to form a first solution;
c) substantially removing the solvent from the first solution to provide a solid product;
d) dissolving the solid product in water to form a second solution;
e) neutralizing the second solution with a base to provide a reaction mass containing paliperidone; and
f) recovering polymorphic Form A2 of paliperidone from the reaction mass.

4. The process of claim 3, wherein the alcoholic solvent is a C₁₋₈ straight or branched chain alcohol selected from the group consisting of methanol, ethanol, n-propanol, isopropanol, n-butanol, isobutanol, tert-butanol, and mixtures thereof.

5. The process of claim 4, wherein the alcoholic solvent is methanol.

6-8. (canceled)

9. The process of claim 3, wherein the hydrochloric acid is in the form of aqueous hydrochloric acid, in the form of hydrogen chloride gas, or in the form of hydrogen chloride dissolved in an alcoholic solvent.

10. (canceled)

11. The process of claim 9, wherein the hydrochloric acid is in the form of methanolic HCl.

12. The process of claim 3, wherein the removing the solvent comprises complete evaporation of the solvent, spray drying, vacuum drying, lyophilization, freeze drying, or a combination thereof.

13. The process of claim 3, wherein the base is an inorganic base selected from the group consisting of ammonia; or hydroxides, carbonates and bicarbonates of alkali or alkaline earth metals.

14. The process of claim 13, wherein the base is selected from the group consisting of ammonia, sodium hydroxide, calcium hydroxide, magnesium hydroxide, potassium hydroxide, lithium hydroxide, sodium carbonate, potassium carbonate, and lithium carbonate.

15. (canceled)

16. The process of claim 3, wherein the pH of the solution in step-(e) is adjusted between 6.5 and 9.

17-20. (canceled)

21. A polymorphic Form A3 of paliperidone characterized by at least one or more of the following properties:
i) a powder X-ray diffraction pattern substantially in accordance with FIG. 4;
ii) a powder X-ray diffraction pattern having peaks at about 5.04, 7.76, 9.62, 10.20, 10.75, 12.02, 13.06, 13.47, 13.89, 14.39, 15.36, 15.66, 16.18, 17.83, 19.07, 19.67, 21.06, 21.41, 22.31, 23.75, 25.00, 25.82, 26.89, 27.2, 30.83, 32.86 and 37.37±0.2 degrees 2-theta substantially as depicted in FIG. 4;
iii) a powder X-ray diffraction pattern having additional peaks at about 12.64, 27.58, 28.12, 30.01, 31.24, 31.75, 32.43, 34.02, 38.81, 39.86, 40.76 and 43.46±0.2 degrees 2-theta substantially as depicted in FIG. 4;
iv) an IR spectrum substantially in accordance with FIG. 5;
v) an IR spectrum having absorption bands at about 2948, 2804, 2761, 1654, 1608, 1537, 1267 and 1121±2 cm⁻¹; and/or
vi) a DSC thermogram substantially in accordance with FIG. 6.

22. A process for the preparation of paliperidone polymorphic Form A3 of claim 21, comprising:
a) providing a suspension of paliperidone in water;
b) combining the suspension with an acid to form a clear solution;
c) neutralizing the clear solution with a base to provide a reaction mass containing paliperidone; and
d) recovering polymorphic Form A3 of paliperidone from the reaction mass.

23-25. (canceled)

26. The process of claim 22, wherein the acid is an organic or inorganic acid; wherein the inorganic acid is selected from the group consisting of hydrochloric acid, hydrobromic acid, sulfuric acid, phosphoric acid, and nitric acid; and wherein the organic acid is selected from the group consisting of p-toluenesulfonic acid, methanesulfonic acid, benzenesulfonic acid, oxalic acid, acetic acid, propionic acid, trifluoroacetic acid, carbonic acid, succinic acid, citric acid, tartaric acid, benzoic acid, maleic acid and fumaric acid.

27. The process of claim 26, wherein the acid is in the form of an aqueous solution.

28. (canceled)

29. The process of claim 26, wherein the inorganic acid is hydrochloric acid.

30-33. (canceled)

34. The process of claim 22, wherein the clear solution is subjected to carbon treatment.

35. The process of claim 22, wherein the base is an organic or inorganic base.

36. The process of claim 35, wherein the base is in the form of an aqueous solution.

37. The process of claim 35, wherein the organic base is selected from the group consisting of triethyl amine, dimethyl amine, tert-butyl amine, diisopropyl amine, dimethyl amine, monomethyl amine, and diisopropyl ethyl amine; and wherein the inorganic base selected from the group consisting of ammonia; hydroxides, carbonates, and bicarbonates of alkali or alkaline earth metals.

38. (canceled)

39. The process of claim 37, wherein the inorganic base is selected from the group consisting of ammonia, sodium hydroxide, calcium hydroxide, magnesium hydroxide, potassium hydroxide, lithium hydroxide, sodium carbonate, potassium carbonate, and lithium carbonate.

40. The process of claim 39, wherein the inorganic base is ammonia or sodium hydroxide.

41-42. (canceled)

43. The process of claim 22, wherein the pH of the solution in step-(c) is adjusted between 6.5 and 9.

44-48. (canceled)

49. A polymorphic Form A4 of paliperidone characterized by at least one or more of the following properties:
i) a powder X-ray diffraction pattern substantially in accordance with FIG. 7;
ii) a powder X-ray diffraction pattern having peaks at about 7.40, 8.15, 9.65, 10.25, 10.78, 12.39, 13.07, 13.80, 14.53, 14.91, 15.71, 16.20, 18.64, 19.14, 19.97, 20.44, 20.66, 21.07, 21.48, 21.99, 24.62, 25.00, 25.83, 27.95, 28.53, 30.72 and 31.15±0.2 degrees 2-theta substantially as depicted in FIG. 7;
iii) a powder X-ray diffraction pattern having additional peaks at about 7.82, 17.58, 17.85, 22.41, 23.15, 23.84, 26.36, 26.95, 29.07, 29.38, 33.60, 35.62, 38.58, 39.50, 39.80 and 42.84±0.2 degrees 2-theta substantially as depicted in FIG. 7;
iv) an IR spectrum substantially in accordance with FIG. 8;
v) an IR spectrum having absorption bands at about 2935, 2786, 2756, 2726, 1630, 1535, 1270 and 1131±2 cm⁻¹; and/or
vi) a DSC thermogram substantially in accordance with FIG. 9.

50. A process for the preparation of paliperidone polymorphic Form A4 of claim 49, which comprises:
a) suspending paliperidone polymorphic Form A3 in water to form a suspension;
b) heating the suspension at a temperature of above about 40° C. for a time period sufficient to convert polymorphic Form A3 into Form A4; and
c) recovering polymorphic Form A4 of paliperidone.

51. The process of claim 50, wherein the paliperidone polymorphic Form A3 used as starting material in step-(a) is prepared as per the process of claim 22.

52. The process of claim 50, wherein heating is at a temperature of about 45° C. to about 80° C. for at least 30 minutes.

53. The process of claim 52, wherein heating is at a temperature of about 50° C. to about 70° C. from about 1 hour to about 8 hours.

54-56. (canceled)

57. A pharmaceutical composition comprising a therapeutically effective amount of any one or a mixture of the paliperidone polymorphic form A2, A3 or A4, and one or more pharmaceutically acceptable excipients.

58. A process for preparing a pharmaceutical composition, comprising combining the paliperidone polymorphic form prepared according to the processes of claim 3, with one or more pharmaceutically acceptable excipients.

59. A process for preparing a pharmaceutical composition, comprising combining the paliperidone polymorphic form prepared according to the processes of claim 22, with one or more pharmaceutically acceptable excipients.

60. A process for preparing a pharmaceutical composition, comprising combining the paliperidone polymorphic form prepared according to the processes of claim 50, with one or more pharmaceutically acceptable excipients.

61. A pharmaceutical composition comprising paliperidone polymorphic Form A2 of claim 1 and one or more pharmaceutically acceptable excipients.

62. A pharmaceutical composition comprising paliperidone polymorphic Form A3 of claim 21 and one or more pharmaceutically acceptable excipients.

63. A pharmaceutical composition comprising paliperidone polymorphic Form A4 of claim 49 and one or more pharmaceutically acceptable excipients.

64. A pharmaceutical composition comprising particles of the paliperidone polymorphic forms A2, A3 or A4, wherein 90 volume-percent of the particles (D₉₀) have a size of less than or equal to about 400 microns and one or more pharmaceutically acceptable excipients.

65. The pharmaceutical composition of claim 64, wherein the 90 volume-percent of the particles (D₉₀) have a size of less than or equal to about 300 microns, less than or equal to about 100 microns, less than or equal to about 60 microns, or less than or equal to about 15 microns.

66-68. (canceled)
